(19) United States
(12) Patent Application Publication
(10) Pub. No.: US 2011/0087093 A1
(43) Pub. Date: Apr. 14, 2011

(54) VALVE CONFIGURATIONS FOR IMPLANTABLE MEDICAL DEVICES

(75) Inventor: Marcia BUISER, Marlborough, MA (US)

(73) Assignee: Navilyst Medical, Inc., Marlborough, MA (US)

(21) Appl. No.: 12/576,927

(22) Filed: Oct. 9, 2009

(51) Int. Cl. A61M 25/14; A61B 6/00

(52) U.S. Cl. 600/435

(57) ABSTRACT

A medical device at least partially insertable into a patient. The device comprises a catheter portion comprising a flexible tube that is at least partially insertable into the patient, and a valve portion proximal to the catheter portion. The valve portion comprises a planar flexible member comprising first and second valve portions separated from one another by an internal slit. The thickness of the planar flexible member at the internal slit is less than the thickness of the planar flexible member at any other location.

### FIELD OF THE INVENTION

The present invention relates to valve configurations used in implantable medical devices.

### BACKGROUND

There are a number of implantable medical devices used for the repeated and prolonged access to a patient's vascular system or other bodily conduits. Such devices include peripherally-inserted central catheters (“PICC's”), central venous catheters (“CVC's”), dialysis catheters, implantable ports, and midline infusion catheters. These devices are typically implanted into a patient for an extended period of time to allow for multiple treatments, such as the delivery of therapeutic agents or dialysis treatments. Use of such devices eliminates the need for multiple placements of single-use devices, thus reducing the risk of infection and placement complications, and reducing the overall cost of patient care. Examples of such implantable medical devices include Vaxcel® PICC's and ports, Xcela® PICC's and ports, and Vaxcel® Plus Chronic Dialysis catheters (all from Navilyst Medical, Inc., Marlborough, Mass.).

Because the aforementioned devices remain in a patient's body for an extended period of time, it is common practice to seal their proximal ends between uses to prevent blood loss and infection. Such a seal may be created with the use of a simple clamp placed on the catheter line, or more recently, with the use of an in-line valve such as that found in the Vaxcel® PICC with PASV® Valve Technology (Navilyst Medical, Inc., Marlborough, Mass.) and described in U.S. Pat. Nos. 5,205,834, 7,252,652, and 7,435,236, which are incorporated herein by reference. In-line valves are pressure activated such that they open to allow for fluid to be delivered to a patient upon the application of some threshold pressure, above which the valve—sometimes in the form of a slit valve—will open, and below which the valve remains closed. These valves are believed to represent improved performance over simple clamps and result in fewer patient complications and infections.

Computed tomography (CT) is increasingly used as a imaging technique for long-term medical patients. Many CT techniques make use of contrast agents to yield high quality images, thus requiring that the contrast agents be administered to the patient prior to the CT imaging. For patients that already have an implanted device that provides access to the vasculature or organ desired to be imaged, it is desirable to use the existing implanted device as a means for administering the contrast agent rather than to make another incision or introduce another catheter line into the patient for this purpose. Given the usual quantity of contrast agent and the short time frame over which it should be administered, however, it is necessary to inject the contrast agent at a relatively high flow rate, such as 5 cc/sec. Not all implantable devices are configured to deliver fluid at this flow rate, or to handle the pressures associated therewith. Some commercial products have recently been developed that use dimensions, configurations, and/or materials that render them suitable for such so-called “power” injections. An example is the Xcela® Power Injectable PICC (Navilyst Medical, Marlborough, Mass.).

In order to use implantable devices that are power injectable and make use of in-line valves, it is necessary to ensure that the valve portion of these devices are capable of handling the flow rates and pressures associated with power injection.

### SUMMARY OF THE INVENTION

In one aspect, the present invention relates to a medical device at least partially insertable into a patient. The device comprises a catheter portion comprising a flexible tube that is at least partially insertable into the patient, and a valve portion proximal to the catheter portion. The valve portion comprises a planar flexible member comprising first and second valve portions separated from one another by an internal slit. The first and second valve portions are configured to move, when subjected to a fluid pressure of at least a predetermined threshold level, to a first open position so that material may flow distally through the valve portion into the catheter portion. The first and second valve portions remain substantially closed at all times when subjected to a fluid pressure less than the threshold level to substantially prevent flow therethrough. The thickness of the planar flexible member at the internal slit is less than the thickness of the planar flexible member at any other location.

In another aspect, the present invention relates to a valve member that is usable within a medical device that is at least partially insertable into a patient. The valve member comprises a planar flexible member comprising first and second valve portions separated from one another by an internal slit. The thickness of the planar flexible member at the internal slit is less than the thickness of the planar flexible member at any other location.

In another aspect, the present invention relates to valve assemblies that incorporate the valve members of the present invention.

In another aspect, the present invention relates to a method of treating a patient by using a medical device of the present invention.

In yet another aspect, the present invention relates to a kit that includes a medical device of the present invention.

### BRIEF DESCRIPTION OF THE DRAWINGS

FIG. 1 is a perspective view of a PICC in an exemplary embodiment of the present invention.

FIG. 2 is a perspective view of the proximal end of an implantable port in an exemplary embodiment of the present invention.

FIG. 3 is an exploded view of a valve assembly that incorporates a valve member of the present invention.

FIG. 4a is a top view of one embodiment of a valve member of the present invention, and FIGS. 4b, 4c, and 4d are side views of various embodiments of valve members of the present invention.

### DETAILED DESCRIPTION

The present invention relates to valve members usable within medical devices, medical devices and valve assemblies that incorporate such valve members, methods of treating patients using such medical devices and valve assemblies, and kits that include such medical devices. While the use of in-line valves such as the slit valves are used in conventional medical devices and therapies, the valves and devices of the present invention make use of configurations that result in beneficial properties, such as the ability to deliver fluids to patients at high pressures and flow rates. This so-called “power injection” may adversely affect current valves that are not designed to be power injectable, such as causing the valve member to become dislodged during use and therefore losing its ability to form a seal over its intended useful lifetime. Although the valve members of the present invention are not limited for use only within power injectable medical devices, the inventor believes that such devices would be particularly benefitted by the valve configurations of the present invention.

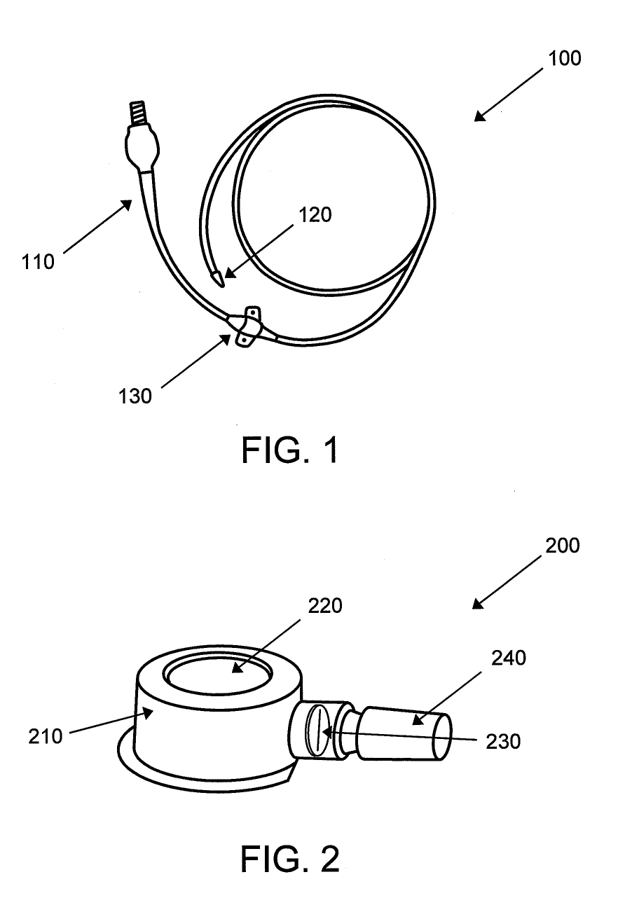

Examples of medical devices that are useful in the present invention include peripherally-inserted central catheters (“PICC's”), central venous catheters (“CVC's”), dialysis catheters, implantable ports, and midline infusion catheters. By way of example, FIG. 1 shows a PICC that makes use of a valve member of the present invention. As shown in FIG. 1, PICC 100 includes a proximal end 110 that, when in use, extends outside of a patient, a distal end 120 that is implanted into the patient's vasculature system, a suture wing 130 for attaching to the patient, and a valve assembly 140 connected to proximal end 110. The distal end 120 (shown curled in FIG. 1) up to the suture wing 130 remains implanted in the patient for an extended period of time for the repeated delivery of therapeutic agents. The in-line valve assembly 140 is used, for example, to seal the PICC so that blood does not flow into the PICC when left in place, and contaminants do not enter the PICC.

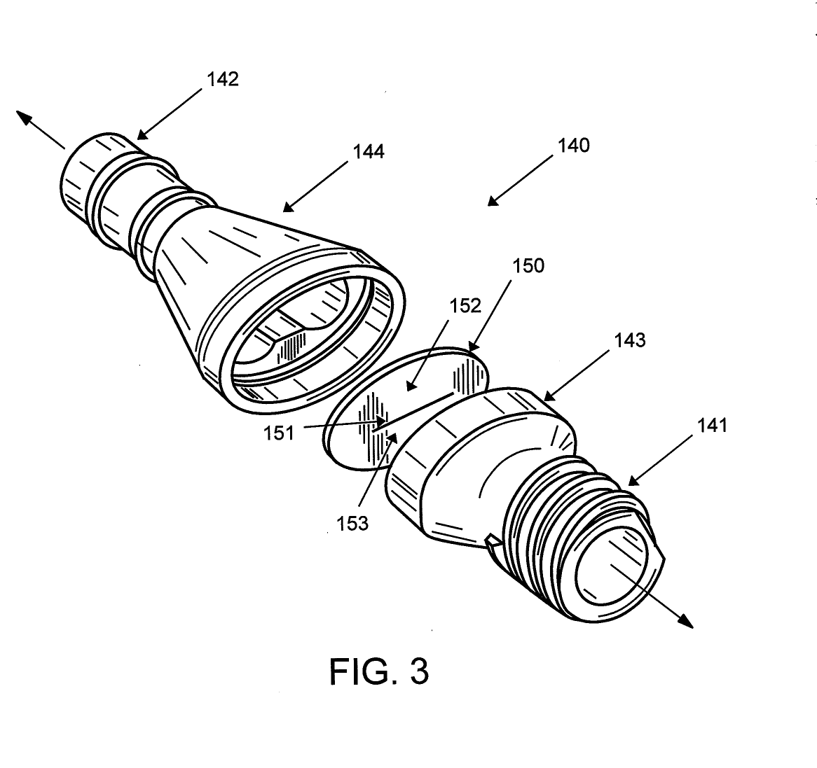

FIG. 2 shows another example of a medical device in the form of an implantable port 200 that makes use of a valve member of the present invention. As is known in the art, the port 200 comprises a housing 210, septum 220, and valve 230. The port 200 is connected to a catheter portion, the proximal end of which is shown at 240. When in use, the port 200 is implanted beneath a patient's skin for an extended period of time for repeated delivery of fluids which are introduced by needle through the skin and septum 220. As with the PICC, the in-line valve 230 is used to create a seal when the port 200 is not being used to deliver fluids to a patient.

An example of a valve assembly 140 that is useful for use in PICCs and other devices of the present invention is described in U.S. Pat. No. 7,252,652, which is incorporated herein by reference. FIG. 3 shows an exploded view of such an assembly, which includes proximal end 141, distal end 142, male housing portion 143, female housing portion 144, and planar, flexible valve member 150. In use, the proximal end 141 is connected to a syringe, IV line, or the like to inject or otherwise deliver fluid to a patient. Such fluids include, for example, therapeutic agents and contrast agents. The distal end 142 is attached as part of a PICC (as shown in FIG. 1) or other suitable device. In the embodiment shown in FIG. 3, the male and female housing portions 143, 144 fit together to house the valve member 150. The valve member 150 includes a slit 151 that is “internal” such that it does not extend to any edge of the valve member 150. The valve member includes first and second valve portions 152, 153 on either side of slit 151. When subjected to a fluid exerted in the distal direction characterized by a pressure of at least a predetermined threshold level, the first and second valve portions 152, 153 move to open the slit in the distal direction so that the fluid may flow distally through the valve member 150 and out the distal end 142 of the housing 140. At pressures lower than this threshold level, the slit remains closed so as to substantially prevent the flow of fluid therethrough. For example, the valves of the present invention remain closed during normal increases in central venous pressure. Whereas the present invention is illustrated as having a single slit 151 within the valve member 150, the invention includes valve members 150 that comprise multiple slits 151 as described herein.

In a preferred embodiment, the valve of the present invention is a two-way valve such that, in addition to opening in a distal direction, it also opens in a proximal direction when subjected to a fluid exerted in the proximal direction characterized by a pressure of at least a predetermined threshold level which may be the same or different from the threshold level required to open the valve in the distal direction. Such two-way valves are useful, for example, to aspirate blood or other bodily fluids for sampling or other purposes.

Suitable materials used to form the valve member 150 include, for example, silicone, rubber, and other elastomeric materials. These materials are formed into the shape of the valve member 150 using any suitable manufacturing technique such as, for example, liquid injection molding, rubber compression molding, and calendaring followed by die cutting.

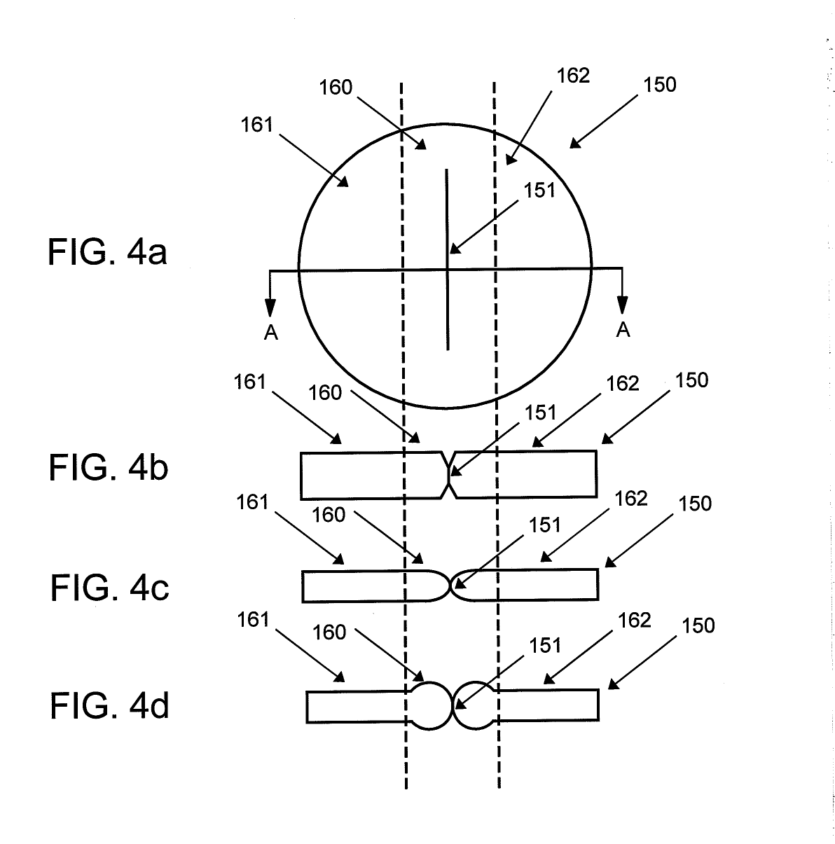

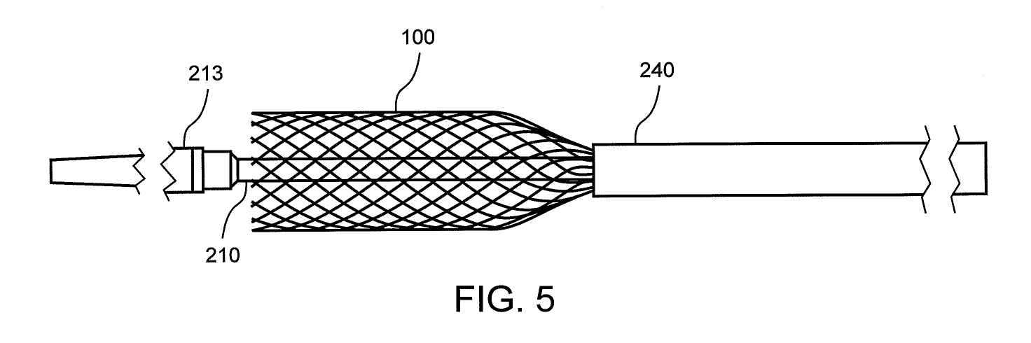

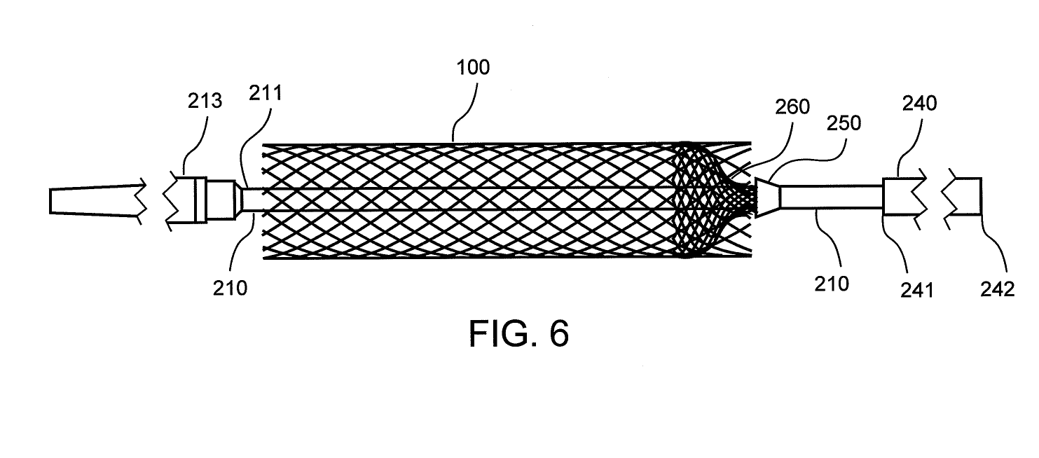

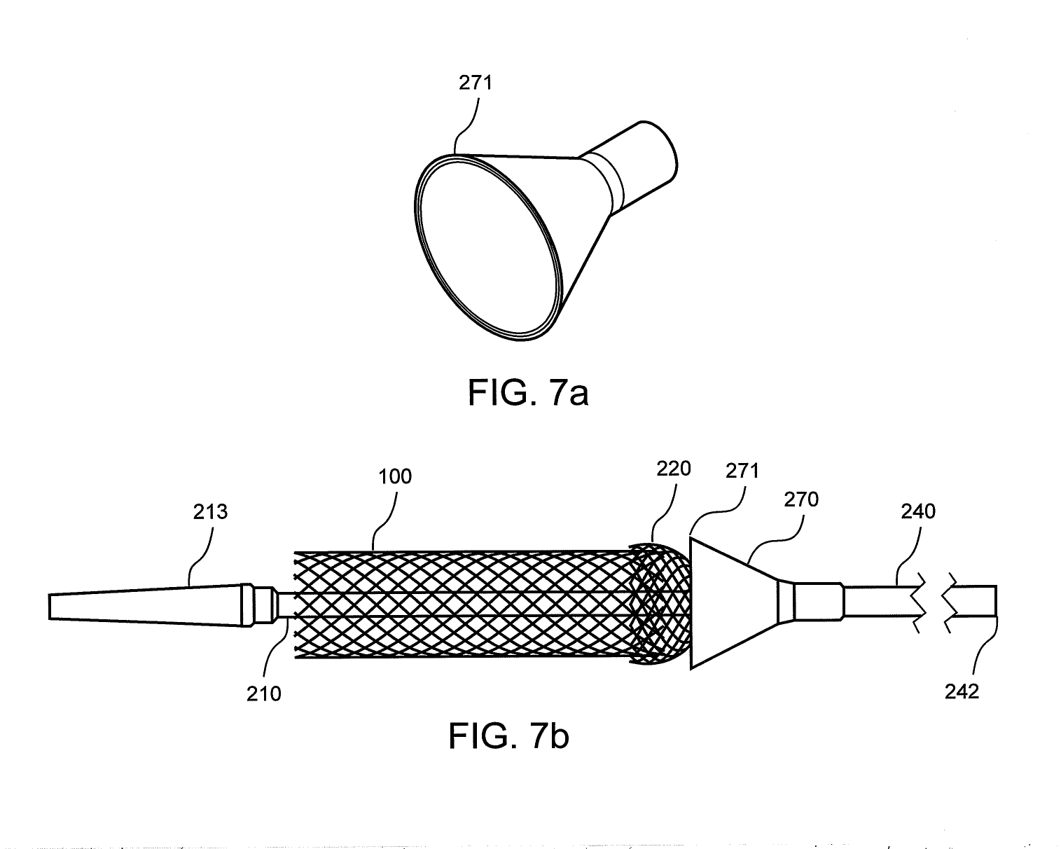

Embodiments of valve configurations within the scope of the present invention are shown in FIGS. 4a through 4d. FIG. 4a shows a top view of a flexible valve member 150, which in this embodiment is a circular disc. In other embodiments, the valve member is of any suitable shape, such as oval, rectangle, or other polygon. Also, whereas the slit 151 is shown in FIG. 4a as a linear slit, the slit may be curved or be of any other suitable configuration.

FIGS. 4b, 4c, and 4d show the cross sectional views of embodiments of the present invention along section AA shown in FIG. 4a. As can be seen from inspection of FIGS. 4b, 4c, and 4d, the thickness of the valve member 150 at the internal slit 151 is less than at any other location along the length of the valve member 150. As an example, the valve member 150 can generally be considered to comprise a central region 160 that includes the internal slit 151, and first and second side regions 161, 162 on either side of the central region 160. In the embodiments shown in FIGS. 4b, 4c, and 4d, the thickness of the first and second side regions 161, 162 are substantially the same, whereas at least a portion of the central region 160 is characterized by a thickness that is less than that of the first and second side regions 161, 162.

By reducing the thickness of the valve member 150 at the location of the slit 151 as compared to the side regions 161, 162, the remainder of the valve member 150 can be constructed with a significantly greater thickness to thereby increase valve strength and yet allow for the necessary opening and closing of the slit 151 during its operation. The increased thickness of the valve member 150 and the associated increased valve strength renders it of particular benefit for power injectable applications. Preferably, the valves and medical devices of the present invention are capable of withstanding fluid injection pressures of greater than about 250 psi, more preferably greater than 300 psi, and most preferably greater than about 325 psi, and fluid flow rates of greater than about 3 cc/sec, more preferably greater than about 4 cc/sec, and most preferably greater than about 5 cc/sec. In a preferred embodiment, the valves and medical devices of the present invention are used to deliver fluid at a rate of about 5 cc/sec at a pressure of about 325 psi.

As shown in FIG. 4b, in one embodiment of the present invention, the valve member 150 is notched in the central region 160 above and below the slit 151. The thicknesses of the valve member 150 in the first and second side regions 161, 162 and at the location of the slit 151 are of any suitable thicknesses to render the valve member 150 useful for its intended purpose and to maximize strength while allowing for full operation of the slit 151. For example, in this embodiment, the thickness of the valve member in the first and second side regions 161, 162 may be within the range of about 0.015-0.020 inches, and preferably about 0.015-0.018 inches for a PICC valve, and within the range of about 0.010-0.014 inches, and preferably about 0.010-0.012 inches for a port valve, which is thicker than that for conventional slit valves used in medical applications; and the thickness at the slit 151 is within the range of about 0.010-0.015 inches, and preferably about 0.013-0.015 inches for a PICC valve, and within the range of about 0.006-0.010 inches, and preferably about 0.008-0.010 inches for a port valve. In other similar embodiments, the valve member 150 is notched only either above or below the slit 151. The embodiment shown in FIG. 4b is manufactured using any suitable manufacturing technique such as, for example, molding followed by a post die-cutting process.

As shown in FIGS. 4c and 4d, in other embodiments of the present invention, the valve member 150 includes rounded edges or arcs to form the slit 151. These arcs are compressed against each other to maintain a tight seal under zero fluid flow conditions, and will roll open in both distal and proximal directions for fluid infusion and aspiration, respectively. As shown in FIG. 4d, the present invention includes embodiments in which the thickness of the valve member 150 in the central region 160 includes at least a portion that is greater than the thickness within the first and second side regions 161, 162. The embodiments shown in FIGS. 4c and 4d are manufactured using any suitable manufacturing technique such as, for example, liquid injection molding. The rounded edges of the embodiments shown in FIGS. 4c and 4d are of any suitable radius of curvature to render the valve member 150 useful for its intended application. As non-limiting examples, the rounded edges of the embodiments shown in FIGS. 4c and 4d form a radius of curvature of about 0.005 inches and 0.010 inches, respectively.

The present invention provides valve configurations that result in enhanced valve properties when compared to conventional in-line medical valves. The present invention may be manufactured, used, or sold as individual valve members for use in fluid delivery devices, as fully assembled housings that include valve members as described herein, or as fully manufactured medical devices.

I claim:

1. A medical device at least partially insertable into a patient, comprising:
a catheter portion comprising a flexible tube at least partially insertable into the patient; and
a valve portion proximal to the catheter portion, the valve portion comprising:
a planar flexible member comprising first and second valve portions separated from one another by an internal slit, the first and second valve portions moving, when subjected to a fluid pressure of at least a predetermined threshold level, to a first open position so that material may flow distally through said valve portion into said catheter portion, the first and second valve portions remaining substantially closed at all times when a fluid pressure exerted thereagainst is less than the threshold level to substantially prevent flow through said valve portion;
wherein the thickness of the planar flexible member at the internal slit is less than the thickness of the planar flexible member at any other location.

2. The medical device of claim 1 wherein, as viewed in a cross-section, the planar flexible member comprises a central region comprising the internal slit, and a first side region and a second side region on respective first and second sides of the central region.

3. The medical device of claim 2, wherein the thickness of the planar flexible member in the first side region is substantially the same as the thickness of the planar flexible member in the second side region.

4. The medical device of claim 3, wherein the planar flexible member is notched in the central region and below the internal slit.

5. The medical device of claim 4, wherein the planar flexible member is notched in the central region and above the internal slit.

6. The medical device of claim 3, wherein each of the first and second valve portions are rounded in the central region to form first and second arcs, respectively, and the first and second arcs contact each other to form the internal slit.

7. The medical device of claim 6, wherein a portion of each of the first and second valve portions in the central region has a thickness that is greater than the thickness of each of the first and second valve portions in the first and second side regions.

8. The medical device of claim 1, wherein the internal slit is formed by cutting through the planar flexible member.

9. The medical device of claim 1, wherein the internal slit is formed by molding the planar flexible member.

10. The medical device of claim 1, wherein the internal slit is substantially linear.

11. The medical device of claim 10, wherein the planar flexible member is formed substantially as a disc and the internal slit extends substantially parallel to a major axis of the disc.

12. The medical device of claim 1, wherein the valve portion includes a plurality of internal slits in the planar flexible member.

13. The medical device of claim 1, wherein said medical device is configured to deliver fluid at a flow rate of at least about 5 cc/sec at a pressure of at least about 300 pounds per square inch.

14. The medical device of claim 13, wherein said fluid is contrast media.

15. The medical device of claim 1, wherein said medical device is a peripherally inserted central catheter, a central venous catheter, a dialysis catheter, an implantable port, or midline infusion catheter.

16. The medical device of claim 1, wherein the first and second valve portions move in a first direction into the first open position in response to pressure along the first direction, and move a second direction to a second open position in response to pressure in the second direction, the first and second directions oriented opposite each other.

17. A medical device at least partially insertable into a patient, comprising:
a catheter portion comprising a flexible tube at least partially insertable into the patient; and
a valve portion proximal to the catheter portion, the valve portion comprising:
a planar flexible member comprising first and second valve portions separated from one another by an internal slit, the first and second valve portions moving, when subjected to a fluid pressure of at least a predetermined threshold level, to a first open position so that material may flow distally through said valve portion into said catheter portion, the first and second valve portions remaining substantially closed at all times when a fluid pressure exerted thereagainst is less than the threshold level to substantially prevent flow through said valve portion;
the planar flexible member, when viewed in cross-section, comprising a central region comprising the internal slit, and a first side region and a second side region on respective first and second sides of the central region, the thickness of the first side region being substantially the same as the thickness of the second side region;
wherein the planar flexible member is notched in the central region and above the internal slit such that the thickness of the planar flexible member at the internal slit is less than the thickness of the planar flexible member at any other location.

18. A medical device at least partially insertable into a patient, comprising:
a catheter portion comprising a flexible tube at least partially insertable into the patient; and
a valve portion proximal to the catheter portion, the valve portion comprising:
a planar flexible member comprising first and second valve portions separated from one another by an internal slit, the first and second valve portions moving, when subjected to a fluid pressure of at least a predetermined threshold level, to a first open position so that material may flow distally through said valve portion into said catheter portion, the first and second valve portions remaining substantially closed at all times when a fluid pressure exerted thereagainst is less than the threshold level to substantially prevent flow through said valve portion;
the planar flexible member, when viewed in cross-section, comprising a central region comprising the internal slit, and a first side region and a second side region on respective first and second sides of the central region, the thickness of the first side region being substantially the same as the thickness of the second side region;
wherein each of the first and second valve portions are rounded in the central region to form first and second arcs that contact each other to form the internal slit such that the thickness of the planar flexible member at the internal slit is less than the thickness of the planar flexible member at any other location.
